(19) United States
(12) Patent Application Publication
(10) Pub. No.: US 2012/0290083 A1
(43) Pub. Date: Nov. 15, 2012

(54) VALVE FOR A HEART VALVE PROSTHESIS

(75) Inventors: Amir Fargahi, Buelach (CH); Matthias Wesselmann, Ruedlingen (CH); Patrice Bachmann, Winterthur (CH); Alwin Schwitzer, Buelach (CH); Bodo Quint, Oberglatt (CH)

(73) Assignee: BIOTRONIK AG, Buelach (CH)

(21) Appl. No.: 13/457,207

(22) Filed: Apr. 26, 2012

Related U.S. Application Data
(60) Provisional application No. 61/484,243, filed on May 10, 2011.

(51) Int. Cl. A61F 2/24

(52) U.S. Cl. 623/2.42

(57) ABSTRACT

A valve for a heart valve prosthesis comprising a valve membrane composed of at least one spiral strip which, in the closed state of the valve membrane, assumes the form of an Archimedean spiral, wherein the outer edge regions of the spiral strip overlap an inner edge region of the spiral strip of a previous winding of the spiral.

### CROSS REFERENCE TO RELATED APPLICATIONS

This patent application claims the benefit of priority to U.S. Provisional Patent Application No. 61/484,243, filed on May 10, 2012, which is herein incorporated by reference in its entirety.

### TECHNICAL FIELD

The invention relates to a valve for a heart valve prosthesis.

### BACKGROUND

The cardiac septum separates the human heart into two halves, i.e. into a right ventricle and a right atrium, and into a left ventricle and a left atrium. Four heart valves are located between the ventricles and the atria. Blood that is anoxemic but rich in carbon dioxide flows first through the tricuspid valve into the right atrium and, from there, into the right ventricle. The tricuspid valve is a tricuspidate valve and is also referred to as an atrioventricular valve. From the right chamber, blood flows through the pulmonary valve into both lungs, where the blood is re-enriched with oxygen. The pulmonary valve is a so-called semilunar valve. The oxygen-enriched blood now leaves the lungs, enters the left atrium, and is pumped through the mitral valve, which has the form of a bicuspidate atrioventricular valve, into the left chamber. Finally, the blood flows out of the left ventricle, through the aortic valve, and into major blood circulation. The aortic valve, similar to the pulmonary valve, is a semilunar valve.

If a patient has heart valve defects, it can be assumed that the functionality of these heart valves can worsen continuously over time. The replacement of heart valves that have stopped functioning with heart valve prostheses has since become second only to the coronary bypass operation as the most common operation performed on the human heart.

In that case, two different types of heart valve prostheses are used, namely mechanical and biological heart valve prostheses.

Biological prostheses are prostheses composed of biological material, in particular the aortic valve leaflets of swine or the pericardium of cattle. For fixation, the tissue is usually chemically treated and attached to a plastic or metal framework for subsequent fixation in the heart.

However, biological heart valve prostheses in the implanted state have a limited service life, because calcification influences the valves to an increasing extent. An average service life of approximately 10 to 12 years is assumed. The rapid calcification of biological heart valves is particularly pronounced in younger patients, however. Biological heart valves are therefore implanted only in patients of advanced age, to avoid the need to replace the valve a second time. Lifelong anticoagulant therapy is required after mechanical heart valve replacement. Compared to biological heart valves, mechanical heart valves have the advantage of a longer service life.

Artificial heart valves are therefore becoming increasingly significant. An ideal heart valve replacement should have an unlimited service life, should allow blood to flow unobstructed in the vessel, should not result in heart valve-related complications such as increased thrombogenicity or susceptibility to endocarditis, should not pose any risks inherent to prostheses, such as valve-related defects, should permit easy implantation, and should be quiet.

Heart valves have since been developed that have a service life of 150 years as demonstrated in the laboratory. The hemodynamic conditions are virtually identical to those of natural heart valves. Valve-related complications such as thrombosis have been virtually eliminated, and excellent continued development has resulted in the practical elimination of prosthesis-related complications. Mechanical heart valves are relatively easy to implant, and the valve noise of the mechanical heart valve is tolerated by the patient relatively well, since it is relatively quiet, at least to the outside world.

Many of the artificial heart valves approved for use at this time require open-heart surgery nearly exclusively for implantation, however, which greatly limits the use of such prostheses, since approximately more than ⅓ of all patients have a high operative risk or are unable to undergo such an operation at all. For this reason, minimally invasive techniques and heart valve prostheses have been developed, in which the new heart valve is delivered to the implantation site using a catheter system and anchored there (e.g. PAVR percutaneous aortic valve replacement). Anchoring in the vessel wall is typically accomplished using a metallic mesh having a design and material selection similar to that of a stent. The mesh can be self-expanding, or can be expanded using a balloon catheter.

Artificial mechanical heart valves intended for use in catheter systems must comprise a valve which is designed for this technology. Solutions to these problems are still very much in demand.

### SUMMARY

The problem to be solved by the present invention is that of reducing or avoiding one or more disadvantages of the prior art. In particular, the problem addressed by the present invention is that of providing a valve for heart valve prostheses, which has been optimized for purposes of catheter-based implantation.

The present invention solves the problem by providing a valve for a heart valve prosthesis comprising a valve membrane composed of at least one spiral strip which, in the closed state of the valve membrane, assumes the form of an Archimedean spiral, wherein the outer edge regions of the spiral strip overlap with an inner edge region of the spiral strip of a previous winding of the spiral.

The invention is based on the idea that a valve membrane—which has been adapted to the special requirements—for a heart valve prosthesis designed for catheter-based, minimally invasive surgery, can be developed using a completely new design of the valve membrane. In that particular case, a spiral strip is shaped such that, overall, a contour of an Archimedean spiral is obtained in the closed state of the valve membrane. The edges of the spiral strip overlap in the direction of blood flow such that each of the inner windings of the spiral comes to rest on a previous winding. It is thereby ensured that the valve can open only in the direction of blood flow. In the opened state, however, the valve membrane assumes the shape of a conical spiral (or a conical, three-dimensional spiral). Blood can s then flow through the exposed opening. The structural solution provided according to the invention is characterized in that, among other things, valve noises can be reduced or prevented. Furthermore, it has the advantage over the previously known designs that it can adapt radially to the size of the vessel. This is particularly suitable for children who are in the growth phase. In addition, the removal/explantation of these heart valve prostheses is simplified.

The valve membrane is affixed to a circumferential valve ring which can be composed of the same material as the membrane. A mesh which is suitable for anchoring in the heart abuts said valve ring in a conventional manner.

The spiral strip is preferably composed of metallic materials such as Nitinol. The surface of the spiral strip can also be coated with ceramic (such as A-SiC), plastic, or an active agent. To simplify explantation of the heart valve prosthesis, it can comprise a marker which is visible to x-rays.

Furthermore, the material can comprise a coating for improving the biocompatibility. The coating contains or is composed of a biocompatible, anorganic material. A biocompatible, anorganic material is a nonliving material that is used for a medical application and interacts with biological systems. A prerequisite for the use of a material that comes in contact with the body environment when used as intended is its biocompatibility. “Biocompatibility” refers to the capability of a material to evoke an appropriate tissue response in a specific application. This includes an adaptation of the chemical, physical, biological, and morphological surface properties of an implant to the recipient tissue, with the objective of achieving a clinically desired interaction. Preferably, biocompatible materials that are substantially bioinert are used for the coating. “Bioinert materials” are those materials that remain substantially intact and exhibit no significant biocorrosion after implantation, for the planned service life of the heart valve prosthesis. Artificial plasma, as prescribed according to EN ISO 10993-15:2000 for biocorrosion assays (composition 6.8 g/l NaCl, 0.2 g/l CaCl₂, 0.4 g/l KCl, 0.1 g/l MgSO₄, 2.2 g/l NaHCO₃, 0.126 g/l Na₂HPO₄, 0.026 g/l NaH₂PO₄), is used as a testing medium to investigate the corrosion behavior of a material under consideration. A sample of the material to be investigated is stored in a closed sample container with a defined quantity of the testing medium at 37° C. The samples are removed and examined in a known manner for traces of corrosion at time intervals defined according to the anticipated corrosion behavior, of a few days up to multiple months or years. The artificial plasma according to EN ISO 10993-15:2000 corresponds to a medium similar to blood and thus represents a possibility for reproducibly simulating a physiological environment within the scope of the invention. A material is considered to be bioinert in particular when the material has corroded by less than 10% in the aforementioned test after a period of 12 months.

Furthermore, the spiral strip preferably has a thickness in the range of 500 to 1,000 micrometers.

The spiral preferably comprises between 2 to 12 windings.

According to another preferred embodiment, an inner end of the spiral strip is designed as a circular disk.

Another aspect of the invention is the provision of a heart valve prosthesis comprising a valve having the aforementioned design.

### DESCRIPTION OF THE DRAWINGS

The invention is explained in greater detail below with reference to embodiments and the related figures. In the drawings:

FIGS. 1A and 1B show a perspective top view and a side view of a valve membrane in the closed state of the valve.

FIGS. 2A and 2B show the same valve membrane as in FIGS. 1A and 1B in a perspective view and a side view, although in the opened state.

FIG. 3 shows a first embodiment of an artificial heart valve prosthesis in the closed state and in the opened state.

is FIG. 4 shows a second embodiment of an artificial heart valve prosthesis in the closed state and in the opened state.

FIG. 5 shows an embodiment of the valve membrane comprising 2 spiral strips.

### DETAILED DESCRIPTION

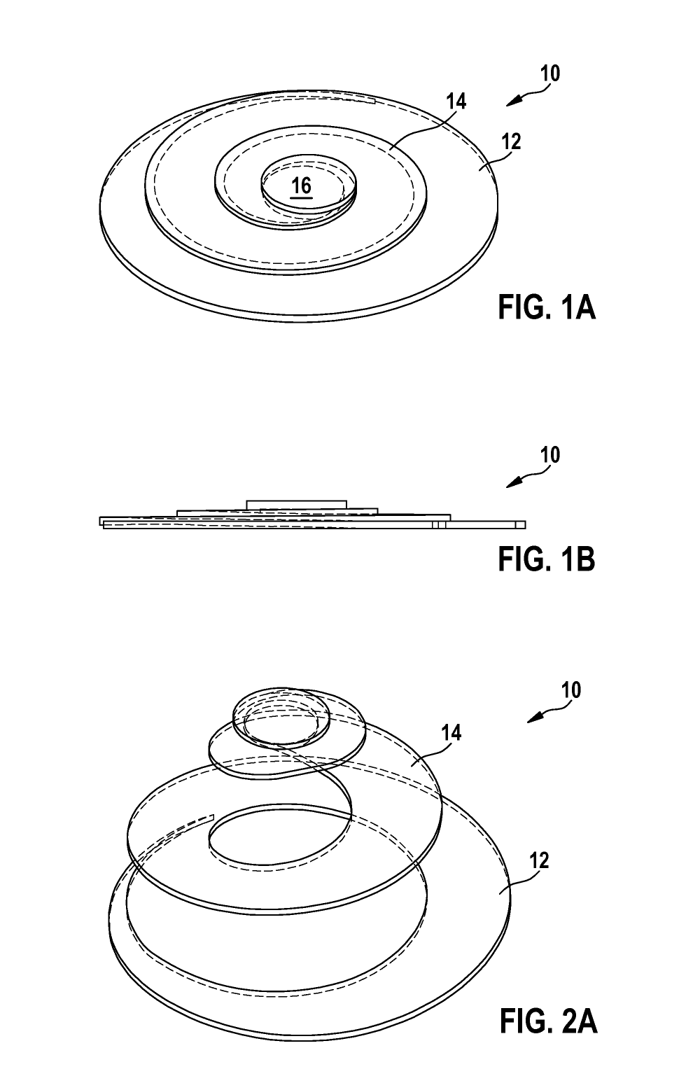

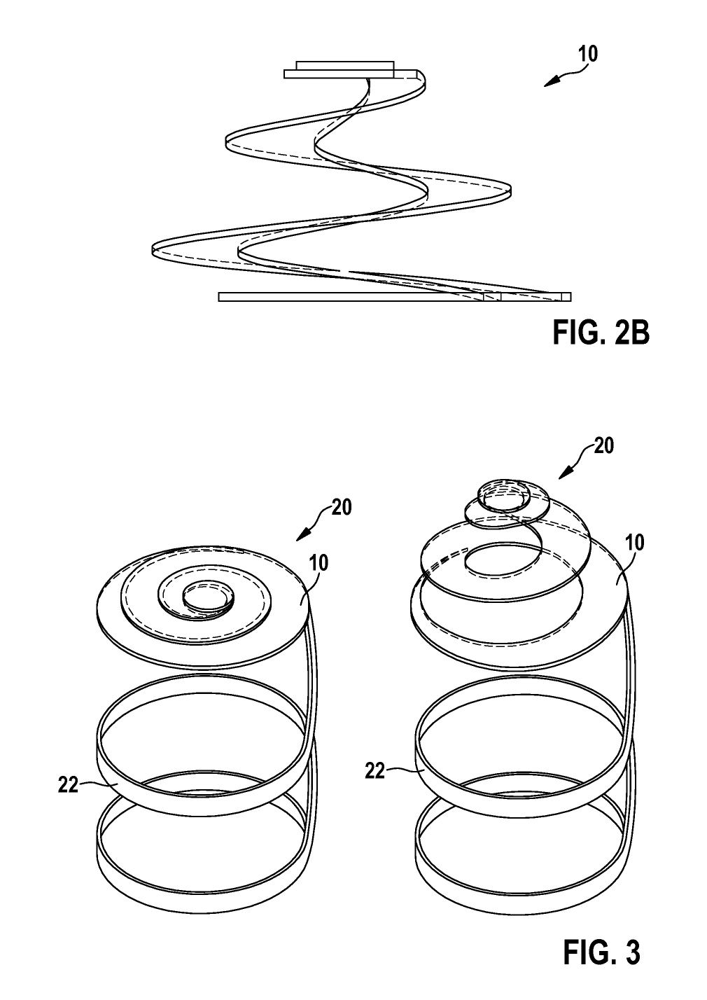

FIGS. 1A to 2B show—each in a different perspective view—a valve membrane 10 designed for use in a valve for a heart valve prosthesis. Valve membrane 10 is shown in the closed state in FIGS. 1A and 1B, and in the opened state in associated FIGS. 2A and 2B in the same perspective view. As shown, valve membrane 10 in the closed state assumes the shape of an Archimedean spiral, the base surface of which is completely closed. For this purpose, a spiral strip 12 is placed on top of one another in a spiral shape such that edge regions 14 of spiral strip 12 overlap in the running direction as the winding increases. According to the embodiment shown, spiral strip 12 leads into a disk-shaped end 16.

The width of the spiral strip can decrease continuously from the outside toward the inside (e.g. from 2.2 mm on the outside to 1.2 mm on the inside). The band strips of the spiral overlap. The overlap can be 0.2 to 0.5 mm, for instance.

Spiral strip 12 can be formed of Nitinol, for instance, and has a thickness of 500 to 1,000 micrometers. The embodiment shown has 3 windings. In the opened state, spiral membrane 10—as shown in FIGS. 2A and 2B—assumes the contour of a conical, three-dimensional spiral (conical spiral) when acted upon by blood flow from the back side.

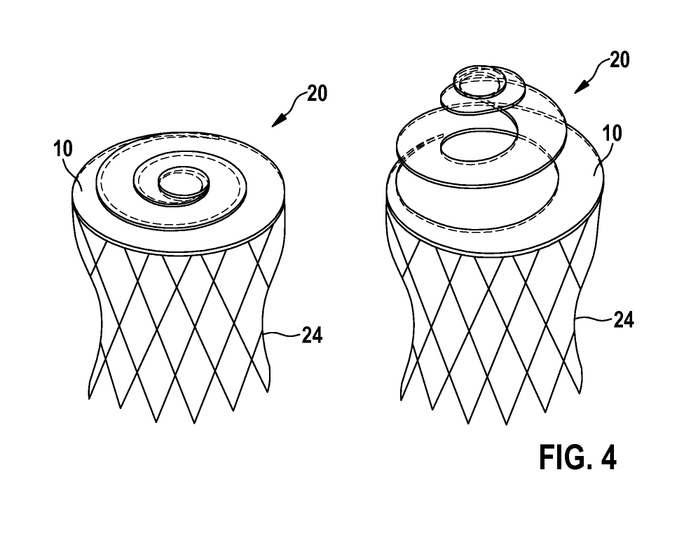

o FIG. 3 shows a heart valve prosthesis 20, in the opened state and closed state, which comprises a valve membrane 10 as depicted in FIG. 1A to 2B. Heart valve prosthesis 20 is anchored at the intended site in the heart using annular fixing elements 22.

Heart valve prosthesis 20 depicted in FIG. 4 also comprises a valve membrane 10 as is shown in FIG. 1A to 2B. In contrast to the embodiment depicted in FIG. 3, affixation takes place here using a metallic mesh 24.

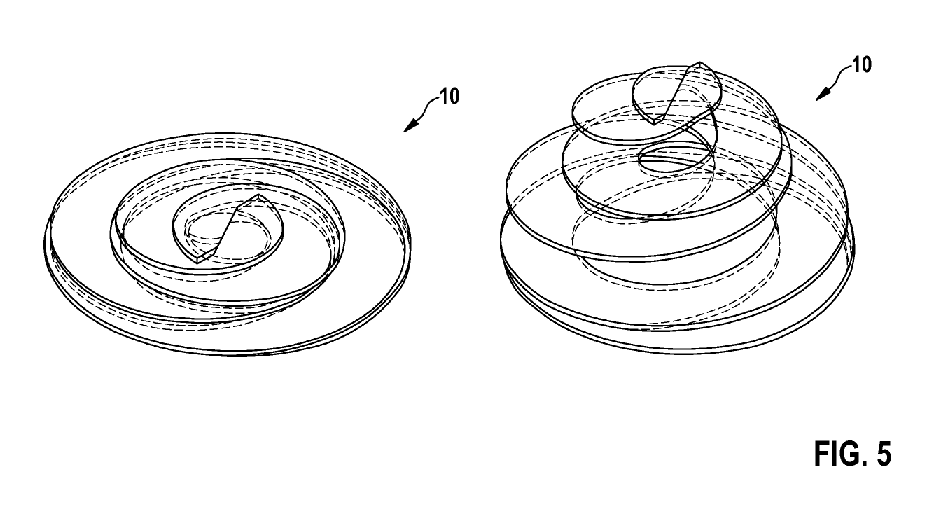

FIG. 5 shows an embodiment of valve membrane 10, in which 2 spiral strips wound into one another come to rest on top of one another in the closed state of the valve such that the entire base surface of the valve membrane is impermeable. In the opened state, the two spiral strips reassume the contour of a conical, three-dimensional spiral.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A valve for a heart valve prosthesis comprising a valve membrane composed of at least one spiral strip which, in the closed state of the valve membrane, assumes the form of an Archimedean spiral, wherein the outer edge regions of the spiral strip overlap an inner edge region of the spiral strip of a previous winding of the spiral.

2. The valve according to claim 1, wherein the valve membrane assumes the shape of a conical spiral in the opened state.

3. The valve according to claim 1, wherein the spiral comprises between 2 to 12 windings.

4. The valve according to claim 1, wherein an inner end of the spiral strip is designed as a circular disk.

5. The valve according to claim 1, wherein the membrane is composed of a metallic material.

6. The valve according to claim 1, wherein the spiral strip has a thickness in the range of 500 to 1,000 micrometers.

7. A heart valve prosthesis comprising a valve according to claim 1.
